(19) United States
(12) Patent Application Publication
(10) Pub. No.: US 2021/0275076 A1
(43) Pub. Date: Sep. 9, 2021

(54) BIOMEDICAL ELECTRODE

(71) Applicant: Welch Allyn, Inc., Skaneateles Falls, NY (US)

(72) Inventors: Shelby Michael Reed, Phoenix, NY (US); Carlos Andres Suarez, Syracuse, NY (US); Thaddeus J. Wawro, Auburn, NY (US)

(21) Appl. No.: 17/188,527

(22) Filed: Mar. 1, 2021

Related U.S. Application Data
(60) Provisional application No. 62/985,485, filed on Mar. 5, 2020.

(51) Int. Cl. A61B 5/266; A61B 5/259; A61B 5/271

(52) U.S. Cl. CPC A61B 5/266; A61B 5/271; A61B 5/259

(57) ABSTRACT

A biomedical electrode includes a hydrogel layer, a trace layer above the hydrogel layer, the trace layer having a conductive trace, a conductive adhesive layer above the trace layer, and a terminal above the conductive adhesive layer. The hydrogel layer, trace layer, conductive adhesive layer, and terminal are assembled together without requiring a piloting effect to overcome misalignment between these components.

### TECHNICAL FIELD

The subject matter described herein relates to a biomedical electrode, for example an electrocardiogram (EKG) electrode, whose manufacture is highly tolerant of misalignment of the electrode components.

### BACKGROUND

Conventional EKG electrodes typically include a hydrogel element, an eyelet positioned on top of and in electrical contact with the hydrogel element, and a stud having an interior cavity. A post portion of the eyelet projects into the interior cavity of the stud to provide a mechanical and electrical connection between the eyelet and stud.

During manufacture of the electrode, the stud and eyelet are aligned with each other and then pressed together to assemble them to each other. The stud and eyelet need to be aligned with each other before pressing them together which can make manufacture of the electrode inefficient by slowing production and increasing manufacturing cost. The need for careful pre-assembly alignment may also reduce the yield of acceptably manufactured electrodes and result in poor quality attachment of the stud to the eyelet.

Moreover, in some cases multiple electrodes may be integrated with a long term wearable device, or may be required to mate with a wearable device such as a processor module or power module as described in U.S. provisional patent application 62/818,271, entitled “Extended Wear Multi-Day Electrode for Vital Sign Patch” filed on Mar. 14, 2019. Because such a device is intended for long term wear, it is desirable for it to be as compact as possible. Unfortunately, the eyelets are large enough that they may to some extent dictate the size of the wearable device. Conversely, if the eyelets can be dispensed with in lieu of alternative components which take less space but retain the function of the electrodes, the goal of compactness may be easier to achieve.

Additionally, the stud needs to be constrained at the same longitudinal and lateral location as the eyelet in order for the eyelet to fit into the interior cavity of the stud, and both need to be constrained within the hydrogel element. Such constraints limit the designer's freedom of design, and may be especially unwelcome when designing devices that integrate multiple electrodes into a single component.

### SUMMARY

In one aspect, a biomedical electrode comprises: a hydrogel layer; a trace layer above the hydrogel layer, the trace layer having a conductive trace; a conductive adhesive layer above the trace layer; and a terminal above the conductive adhesive layer.

In another aspect, a biomedical electrode suite comprises: a hydrogel layer comprising first and second hydrogel elements; a trace layer above the hydrogel layer, the trace layer including a first trace in electrical contact with the first hydrogel element and a second trace in electrical contact with the second hydrogel element; a conductive adhesive layer above the trace layer; the conductive adhesive layer having a first conductive portion in electrical contact with the first trace and a second conductive portion in electrical contact with the second trace; and a terminal array including a first terminal above and in contact with the first conductive portion of the conductive adhesive layer and a second terminal above and in contact with the second conductive portion of the conductive adhesive layer; wherein the first hydrogel element, the first trace, the first conductive portion, and the first terminal define a first electrical path, and the second hydrogel element, the second trace, the second conductive portion, and the second terminal define a second electrical path separate from the first electrical path.

In another aspect, a method of assembly for an electrode comprises: providing a hydrogel layer; assembling a trace layer to the hydrogel layer; assembling a conductive adhesive layer to the trace layer; assembling a terminal to the conductive adhesive layer; wherein assembling the trace layer, the conductive adhesive layer, and the terminal is performed without a piloting effect.

### BRIEF DESCRIPTION OF THE DRAWINGS

The following drawing figures, which form a part of this application, are illustrative of the described technology and are not meant to limit the scope of the disclosure in any manner. The drawings are not drawn to scale.

FIG. 1 is an exploded view of a conventional electrocardiogram (EKG) electrode having an eyelet and a stud.

FIG. 2 is a cross sectional elevation view of the electrode of FIG. 1.

FIGS. 3A, 3B, 3C, and 3D are a sequence of cross sectional side elevation views showing the assembly of the stud to the eyelet of the electrode of FIG. 1 is tolerant of minor misalignment of the stud and eyelet axes.

FIGS. 4A and 4B are cross sectional side elevation views showing that assembly of the stud to the eyelet of the electrode of FIG. 1 is not tolerant of other than minor misalignment of the stud and eyelet axes.

FIGS. 5 and 6 are exploded and side elevation views, respectively, showing an electrode that is more tolerant to misalignment of its components, in accordance with the embodiment described herein.

FIGS. 7, 8, 9, 10 and 11 are side elevation views showing electrical continuity of the electrode of FIGS. 5 and 6 survives a misalignment of its components.

FIG. 12 is an exploded view of a biomedical electrode suite having multiple electrodes, each analogous to the single electrode of FIG. 5.

FIG. 13 is a plan view showing the bottom of a trace layer of the electrode suite of FIG. 12.

FIG. 14 is a cross sectional side elevation view of the electrode suite of FIG. 12 in its assembled state.

FIGS. 15, 16, 17 and 18 are side elevation views showing electrical continuity of the electrode suite of FIGS. 12-14 survives a misalignment of its components.

### DETAILED DESCRIPTION

Various embodiments will be described in detail with reference to the drawings, wherein like reference numerals represent like parts and assemblies throughout the several views. Reference to the various embodiments does not limit the scope of the claims attached hereto. Additionally, any examples set forth in this specification are not intended to be limiting for the appended claims.

The terms “substantially” and “about” may be used herein to represent the inherent degree of uncertainty that may be attributed to any quantitative comparison, value, measurement or other representation. These terms may also be used herein to represent the degree by which a quantitative representation may vary from a stated reference without resulting in a change in the basic function of the subject matter at issue.

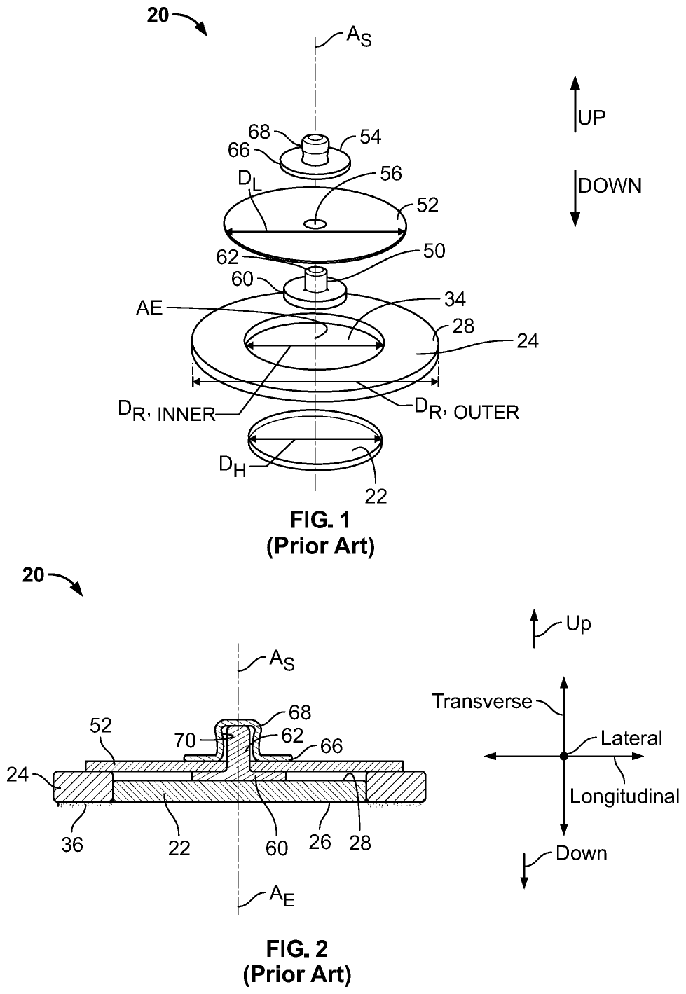

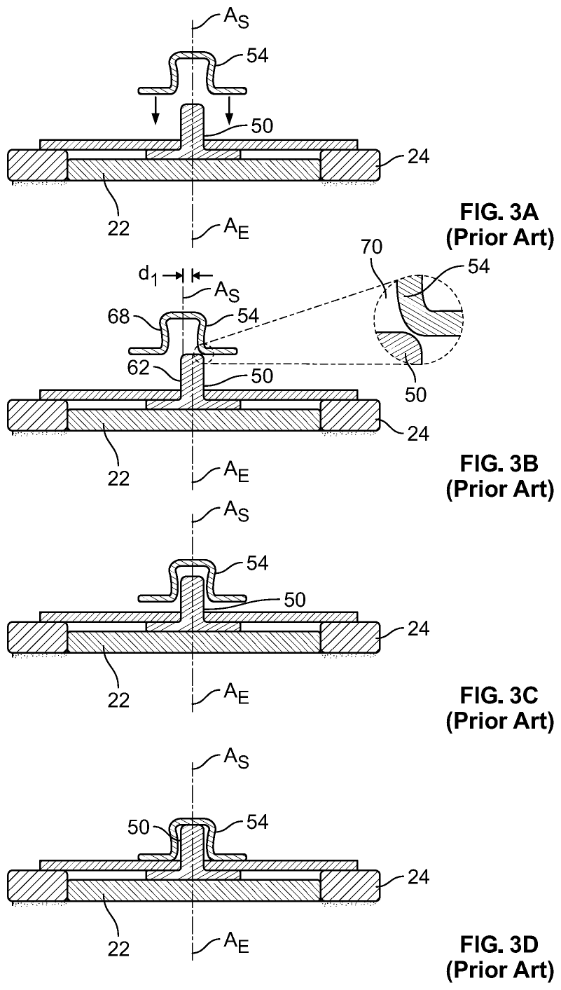

FIGS. 1 and 2 illustrate an electrode 20, which is a conventional biometric electrode such as an electrocardiogram electrode. The figures include longitudinal, lateral, and transverse reference axes. In this specification the transverse direction may be referred to as the vertical direction, with UP and DOWN as indicated by the arrows so labeled. However it should be understood that the following description of the prior art electrode and Applicant's electrode are independent of orientation.

The electrode 20 includes a hydrogel element 22 having a diameter DH. In the example shown in FIGS. 1 and 2, the hydrogel element 22 has a circular disk shape.

The electrode 20 includes an enclosure element 24 that circumscribes the hydrogel element 22. The enclosure element 24 has a lower side 26 and an upper side 28. When the electrode 20 is attached to a patient's body, the lower side 26 faces the patient's body while the upper side 28 faces away from the patient's body.

The enclosure element 24 has an outer diameter D_{R,OUTER}. An opening 34 in the enclosure element 24 has a diameter D_{R,INNER} which is about the same as or slightly less than diameter DH of the hydrogel element 22. This allows the hydrogel element 22 to nest securely inside the opening 34 of the enclosure element 24.

An adhesive 36 resides on the lower side 26 of the enclosure element 24 such that the electrode 20 can adhere to the patient's body. A release liner, not illustrated, may be applied to the adhesive 36. A caregiver can peel the release liner off the electrode 20 to expose the adhesive 36 to adhere the electrode 20 to the patient's body.

The electrode 20 further includes an eyelet 50 having a base 60 and an eyelet post 62, a label 52 with a central opening 56, and a terminal in the form of a stud 54. The stud 54 includes a base 66 and a pedestal 68. The pedestal 68 is hollow, thereby defining a pedestal interior cavity 70. The eyelet 50 rests on top of the hydrogel element 22 with the eyelet post 62 projecting through central opening 56 of the label 52.

The label 52 has a label diameter DL that is greater than the hydrogel element diameter DH and less than the D_{R,OUTER}. This results in a radially outer portion of the label 52 residing atop the enclosure element 24. As shown in FIG. 2, the base 66 of the stud 54 resides on top of the label 52 such that the pedestal interior cavity 70 receives the eyelet post 62 thereby attaching and electrically connecting the stud 54 to the eyelet 50.

In use, a caregiver attaches wires from monitoring equipment to the pedestal 68. Electrical signals from the patient propagate to the monitoring equipment by way of the hydrogel element 22, the eyelet 50, and the stud 54.

The eyelet 50 has an eyelet axis AE. The stud 54 has an axis A_{S} which is depicted less boldly than the eyelet axis AE to assist the reader in distinguishing the axes from each other. As shown in FIG. 2, when the electrode 20 is assembled, the geometry of eyelet post 62 (which is cylindrical or nearly so) and the geometry of the pedestal interior cavity 70 (which is also cylindrical or nearly so) constrain the axes A_{E}, A_{S} to be coaxial, a condition which may also be referred to herein as “on-axis” or aligned.

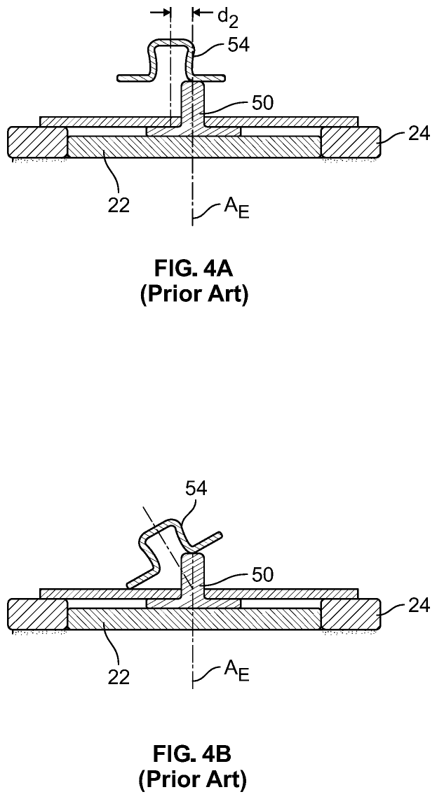

During manufacture and assembly of the electrode 20, the stud 54 must be well aligned with the eyelet 50 in order to mate the stud 54 with the eyelet 50, as shown in FIGS. 1 and 2. This is further illustrated in the sequence of views of FIGS. 3A-3D.

In FIG. 3A, the stud 54 is lowered toward the eyelet 50. In FIG. 3B, the stud 54 begins to engage the eyelet 50. As shown in FIGS. 3C and 3D, despite the slight misalignment represented by offset d₁ between axes A_{S} and A_{E}, the stud 54 can receive the eyelet post 62. This is because continued downward movement of the stud 54 pilots the stud 54 to the right thereby centering the stud 54 on the eyelet 50 (specifically centering the pedestal interior cavity 70 of the stud 54 on the eyelet post 62).

One illustrative example for a standard for judging whether the stud 54 is well aligned with the eyelet 50, (i.e. that the misalignment d₁ between their axes A_{E}, A_{S} is small) is that, despite the misalignment, assembly of the electrode 20 can nevertheless proceed due to a piloting effect that results from the fact that the misalignment d₁ is overcome by the engagement of the eyelet post 62 and the interior surface of the stud 54 once they come into contact and interact with each other (e.g., see FIG. 3B).

An alternative standard for judging whether the stud 54 is well aligned with the eyelet 50 is that the misalignment is correctable by the engagement of the stud 54 with the eyelet 50. The ability to overcome the misalignment is facilitated by features such as rounded corners on the stud 54 and eyelet 50 as shown in the inset to FIG. 3B.

As used herein, being subject to a piloting effect includes providing for a piloting effect even though some sets of components may be well enough aligned before being brought together that little or no piloting occurs as the result of the components engaging with each other. For example, when conventional electrodes such as the shown in FIG. 1 are mass-produced, the components may sometimes be well enough aligned that no piloting occurs. Nevertheless, because such perfection of alignment cannot be assured for all electrodes being manufactured, at least some components are designed to cause piloting during assembly such as by the rounded corners on the stud 54 and eyelet 50 described in connection with the inset to FIG. 3B). Therefore, all of the electrodes are “subject to a piloting effect” even though not all will actually require it.

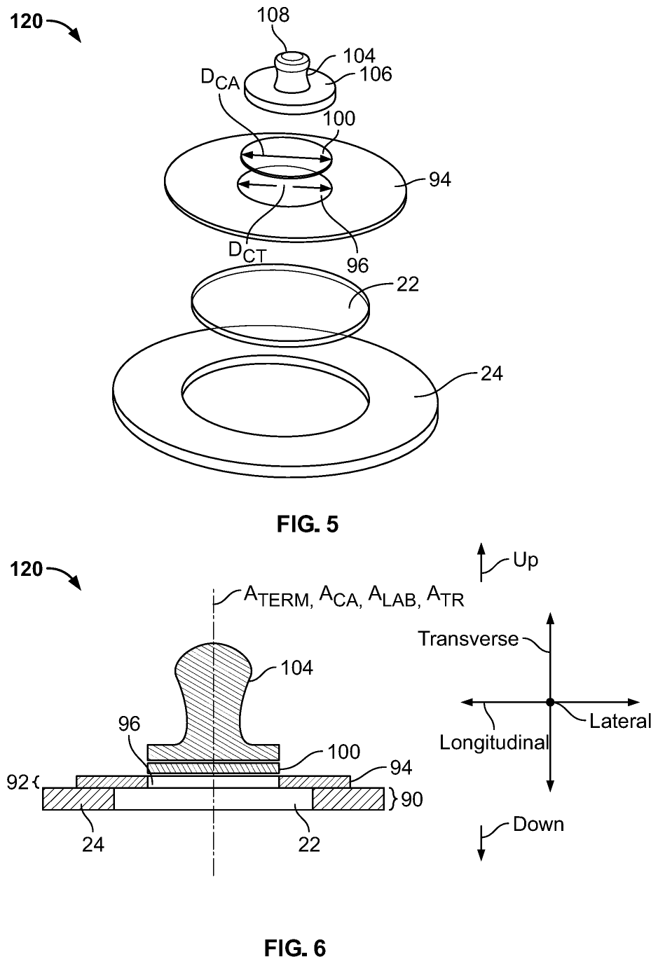

In contrast to the misalignment d₁ of FIG. 3B, the misalignment d₂ of FIGS. 4A and 4B is larger. As shown in FIGS. 4A and 4B, the larger misalignment causes the stud 54 to rock to the left rather than being piloted to an on-axis or aligned state as in FIG. 3D. In view of the foregoing, only small misalignments are tolerable for the assembly of the stud 54 and the eyelet 50, as shown in FIGS. 3A and 3B.

The required precision of alignment is contrary to the desire to make the manufacturing process as quick, inexpensive, efficient, and high yield as possible. Further manufacturing difficulty arises from the fact that the eyelet 50 must be provided from beneath the label 52 whereas the stud 54 must be provided from above the label.

Additionally, because the eyelet 50 must be able to fit into the pedestal interior cavity 70 of the stud 54, the stud 54 is constrained to be at the same longitudinal and lateral location as the eyelet 50, which itself must be within the planform of the hydrogel element 22. Such constraints limit the freedom to design a device such as an electrode suite that integrates multiple electrodes into a single device.

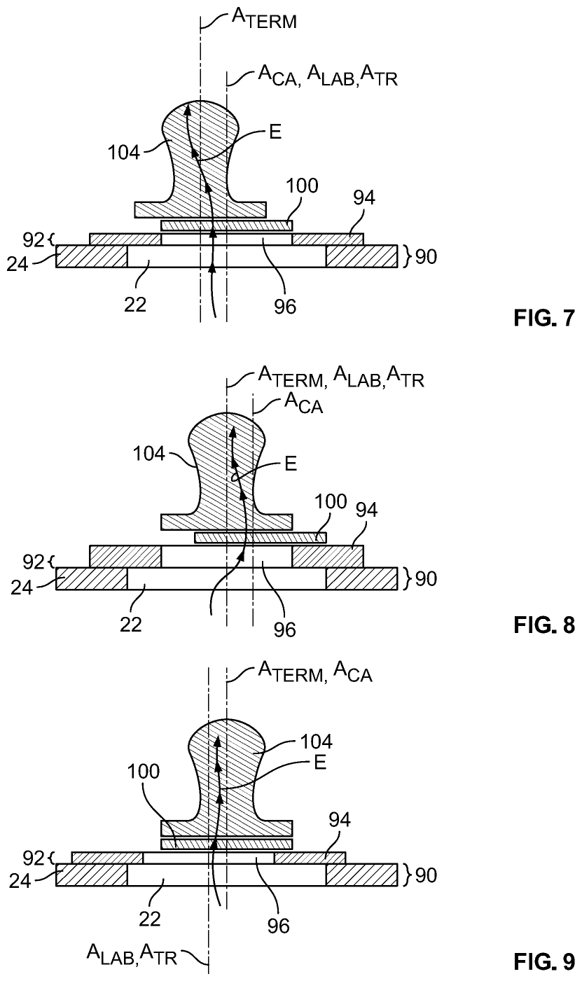

FIGS. 5 and 6 show an improved embodiment of an electrode 120 which is simpler to produce. The electrode 120 includes a hydrogel layer 90 having a hydrogel element 22 and an enclosure element 24 which circumscribes the hydrogel element. In certain examples, the enclosure element 24 is made from a foam material.

The electrode 120 includes a trace layer 92 above the hydrogel layer 90. The trace layer 92 includes a label 94 and a conductive trace 96. In the illustrated examples, the conductive trace 96 is a circular patch. The diameter of the conductive trace 96 is D_{CT}. In some examples, the conductive trace 96 is made of conductive ink which may be printed onto the label 94. In certain examples, the conductive ink is an Ag—AgCl ink.

The electrode 120 includes an conductive adhesive layer 100 above the trace layer 92. The conductive adhesive layer 100 is a patch of adhesive. In the example illustrated in FIG. 5, the conductive adhesive layer 100 has a circular profile that has a diameter D_{CA} that is approximately equal to diameter D_{CT} of the conductive trace 96.

The electrode 120 further includes a terminal 104 having a base 106 and a pedestal 108. The terminal 104 may be thought of as a terminal layer. Unlike the pedestal 68 of the stud 54 of FIGS. 1 and 2, the pedestal 108 of FIGS. 5 and 6 need not be hollow. The terminal 104 rests on top of the conductive adhesive layer 100.

The conductive adhesive layer 100 provides a mechanical bond between the terminal 104 and the conductive trace 96. Due to its electrical conductivity, the conductive adhesive layer 100 also provides electrical connectivity between the stud 54 and the conductive trace 96. The conductive adhesive layer 100 also acts as a gasket which seals the hydrogel element 22 from fluid ingress during an extended wear interval when the patient bathes or showers and therefore exposes the electrode 120 to water.

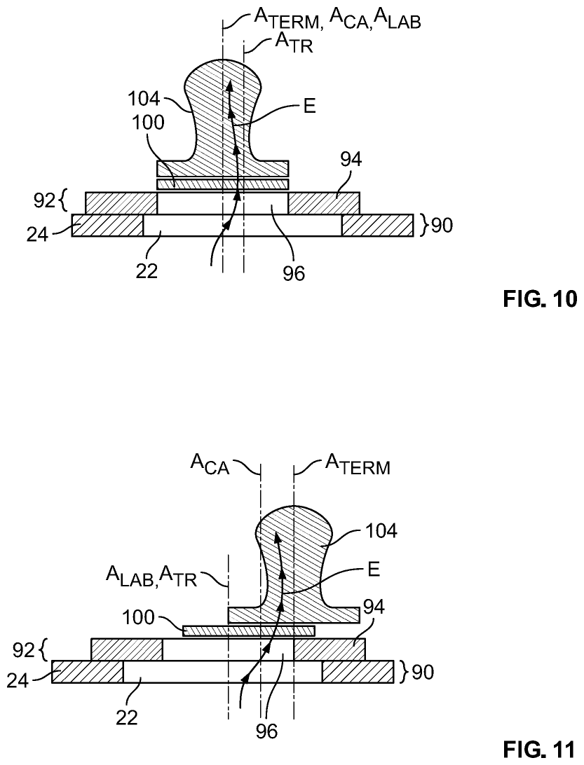

Referring now to FIGS. 7-11, the terminal 104 has a terminal axis A_{TERM}, the conductive adhesive layer 100 has a conductive adhesive layer axis A_{CA}, the label 94 has a label axis A_{LAB}, and the trace layer 92 has a trace axis A_{TR}. In an ideal assembly, the axes A_{TERM}, A_{CA}, A_{LAB}, and A_{TR} are substantially aligned with each other as in FIGS. 5 and 6. Advantageously, electrical continuity from the hydrogel element 22 to the terminal 104 survives at least two of the terminal axis A_{TERM}, the conductive adhesive layer axis A_{CA}, the label axis A_{LAB}, and the trace axis A_{TR}, being offset from each other without requiring a piloting effect to overcome the misalignment.

FIG. 7 shows the terminal 104 offset from the other components. FIG. 8 shows the conductive adhesive layer 100 offset from the other components. FIG. 9 shows trace layer 92 offset from the other components. In FIG. 9, the label 94 and the conductive trace 96 are not offset relative to each other such that the trace layer 92 is offset as a whole without any effect on the relationship of its sub-elements (i.e., the label 94 and the conductive trace 96). FIG. 10 shows the conductive trace 96 off-center on the label 94. In FIG. 10, the trace layer 92 as a whole is not offset relative to the hydrogel element 22, the conductive adhesive layer 100, or the terminal 104. In all four illustrative examples shown in FIG. 7-10, no piloting effect is required during manufacture in order to bring the four axes A_{TERM}, A_{TR}, A_{CA}, A_{LAB}, or any two of them, into alignment.

Advantageously, multiple layers of the electrode 120 can be offset relative to each other. For example, FIG. 11 shows two components, such as the terminal 104 and the conductive adhesive layer 100, that are offset relative to each other and that are also offset relative to the trace layer 92 and the hydrogel element 22.

Referring now to FIGS. 7-11, assembly of the components of the electrode 120, such as the terminal 104, the conductive adhesive layer 100, and the trace layer 92, is not subject to a piloting effect. The components of the electrode 120 do not include any physical features to correct any misalignment, whether that misalignment is large or small. Any misalignment(s) that are present as the components of the electrode 120 are assembled together will remain present in the assembly of those components, and a piloting effect is not needed to ensure electrical continuity. For example, the arrow E in FIGS. 7-11 shows that electrical continuity is preserved through the components of the electrode 120, despite misalignments between the components.

As an example, assembly of the terminal 104 to the conductive adhesive layer 100 is not subject to a piloting effect to engage the terminal 104 and the conductive adhesive layer 100 with each other, nor is such piloting required. There are no features that would cause such piloting to occur. The electrode 120 does not include any physical features to correct any misalignment between the terminal 104 and conductive adhesive layer 100, whether large or small. Any misalignment that is present as the terminal 104 and the conductive adhesive layer 100 are assembled together will remain present in the assembly of the terminal 104 and the conductive adhesive layer 100.

Likewise, assembly of the conductive adhesive layer 100 to the trace layer 92 is not subject to a piloting effect that would cause the conductive adhesive layer 100 and the trace layer 92 to engage with each other, nor is such piloting required. There are no features that would cause such piloting to occur. The electrode 120 does not include any physical feature(s) to correct any misalignment between the conductive adhesive layer 100 and trace layer 92, whether large or small. Any misalignment that is present as the conductive adhesive layer 100 and the trace layer 92 are assembled together will remain present in the assembly of the conductive adhesive layer 100 and the trace layer 92.

Assembly of the trace layer 92 to the hydrogel element 22 is not subject to a piloting effect that would cause the trace layer 92 and the hydrogel element 22 to engage with each other, nor is such piloting required. There are no features that would cause such piloting to occur. The electrode 120 does not include any physical feature(s) to correct any misalignment between the trace layer 92 and hydrogel element 22, whether large or small. Any misalignment as the trace layer 92 and hydrogel element 22 are assembled together will remain in the assembly of the trace layer 92 and hydrogel element 22.

Unlike a conventional electrode, such as the electrode 20 shown in FIGS. 1 and 2 in which the stud 54 and eyelet 50 are brought together from opposite sides of the label 52, the electrode 120 of FIGS. 5 and 6 can be assembled by sequentially layering the electrode components on top of one another. For example, manufacturing could begin by providing the hydrogel layer 90, then layering the trace layer 92 on top of the hydrogel layer 90, then layering the conductive adhesive layer 100 on top of the trace layer 92, and then applying the terminal 104 to the top of the conductive adhesive layer 100.

Some degree of alignment of the layers is necessary irrespective of whether the buildup of layers involves assembly of distinct components, printing, laminating, or some combination of these techniques. However the required alignment accuracy is less stringent than the stud/eyelet alignment accuracy demanded when assembling the stud 54 and eyelet 50 of a conventional electrode such as the electrode 20 of FIGS. 1 and 2.

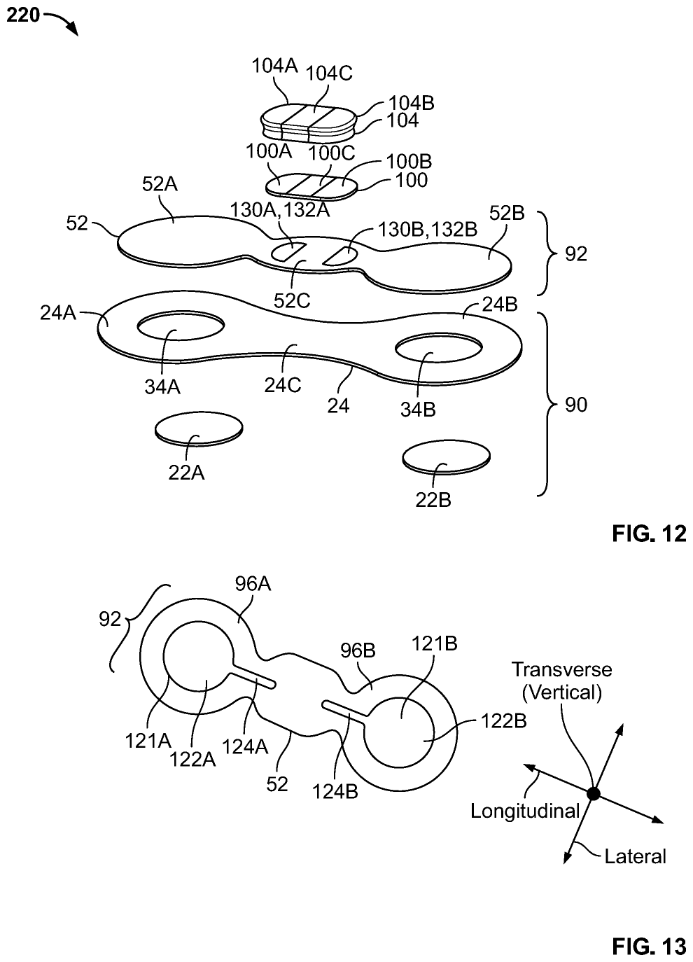

FIGS. 12-14 show a biomedical electrode suite 220 having multiple electrodes analogous to electrode 120. Referring now to FIGS. 12-14, the electrode suite 220 includes a hydrogel layer 90 comprising first and second hydrogel elements 22A, 22B, each in the form of a hydrogel element. The hydrogel layer 90 further includes an enclosure element 24 having first and second end portions 24A, 24B connected to each other by a bridge portion 24C such that the enclosure element 24 has a “dog bone” shape. Openings 34A, 34B penetrate through the first and second end portions 24A, 24B such that each end portion can circumscribe one of the first and second hydrogel elements 22A, 22B. In certain examples, the enclosure element 24 is made from a foam material.

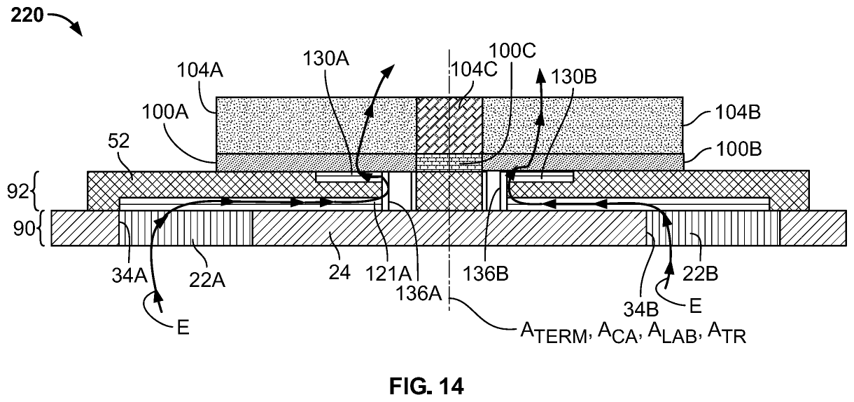

The electrode suite 220 includes a trace layer 92 above the hydrogel layer 90. Trace layer 92 includes a label 52 having a shape and size that correspond to that of the enclosure element 24. For example, the label 52 has outboard portions 52A, 52B connected to each other by an inboard portion 52C such that the label 52 has a similar “dog bone” shape as the enclosure element 24. As shown in FIG. 13, the trace layer 92 further includes a first trace 96A in electrical contact with the first hydrogel element 22A, and a second trace 96B in electrical contact with the second hydrogel element 22B.

As shown in FIG. 13, each of the first and second traces 96A, 96B includes a lower section 121A, 121B comprising a contact element 122A, 122B and a spur 124A, 124B extending from the contact element 122A, 122B to inboard portion 52C of the label 52. The contact element 122A, 122B and spurs 124A, 124B are exposed on a bottom surface of the trace layer 92, but not on a top surface of the trace layer 92. In the assembled electrode suite 220, each contact element 122A, 122B is in contact with a corresponding hydrogel element of the first and second hydrogel elements 22A, 22B.

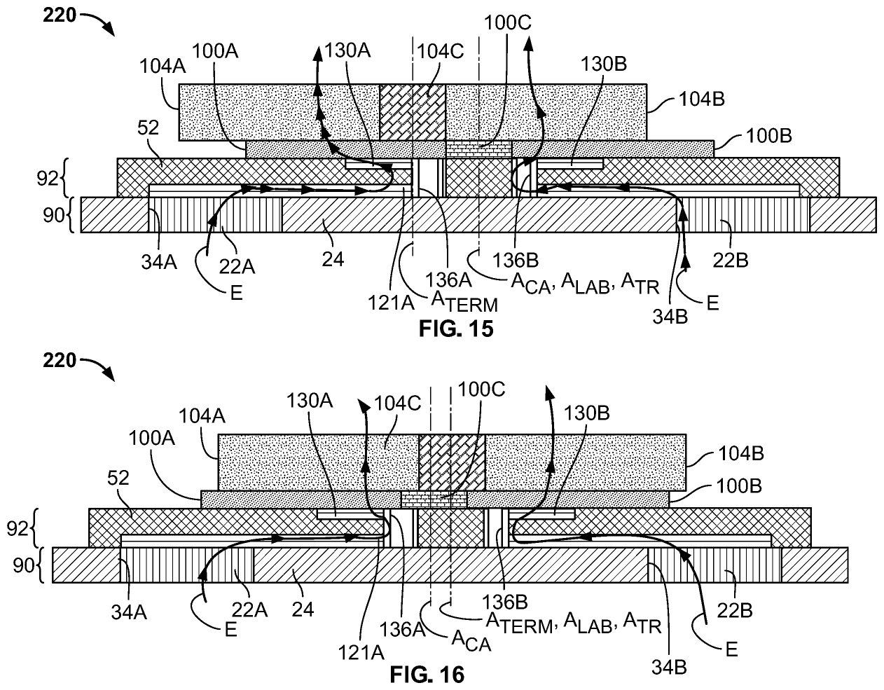

Each of the first and second traces 96A, 96B also includes an upper section 130A, 130B comprised of an upper contact element 132A, 132B which is exposed on the top surface of the trace layer 92, but not on the bottom surface of the trace layer 92. As shown in FIG. 14, each of the first and second traces 96A, 96B includes an electrical vertical interconnect access 136A, 136B that provides electrical continuity between the bottom and upper surfaces of the trace layer 92.

In some examples, each of the each of the first and second traces 96A, 96B is formed by a conductive ink. In certain examples, the conductive ink is an Ag—AgCl ink, which may be printed onto the label 52.

The electrode suite 220 further includes an conductive adhesive layer 100 above the trace layer 92. The conductive adhesive layer 100 includes a first conductive portion 100A in electrical contact with the first trace 96A and a second conductive portion 100B in electrical contact with the second trace 96B. More specifically, the first conductive portion 100A is in electrical contact with the upper contact element 132A of the first trace 96A, and the second conductive portion 100B is in electrical contact with the upper contact element 132B of the second trace 96B.

A nonconductive spacer 100C separates the first and second conductive portions 100A, 100B to electrically isolate them from each other. This provides two separate electrical paths, one for each of the first and second hydrogel elements 22A, 22B. As with the electrode 120, the conductive adhesive layer 100 also acts as a gasket which seals the first and second hydrogel elements 22A, 22B from fluid ingress during an extended wear interval that exposes the electrode suite 220 to water such as when a patient who is wearing the electrode suite 220 bathes or showers.

The electrode suite 220 also includes a terminal array 102 that includes a first terminal 104A above and in contact with first conductive portion 100A of the conductive adhesive layer 100 and a second terminal 104B above and in contact with the second conductive portion 100B of the conductive adhesive layer 100. The terminal array 102 may be considered a terminal layer, or simply a terminal.

In the examples illustrated in the figures, the terminal array 102 includes a nonconductive medial portion 104C, for example a nonconductive plastic, that is integrated with the first and second terminals 104A, 104B to electrically separate the terminals from each other so that each terminal is associated with only one of the two electrical paths originating at the first and second hydrogel elements 22A, 22B.

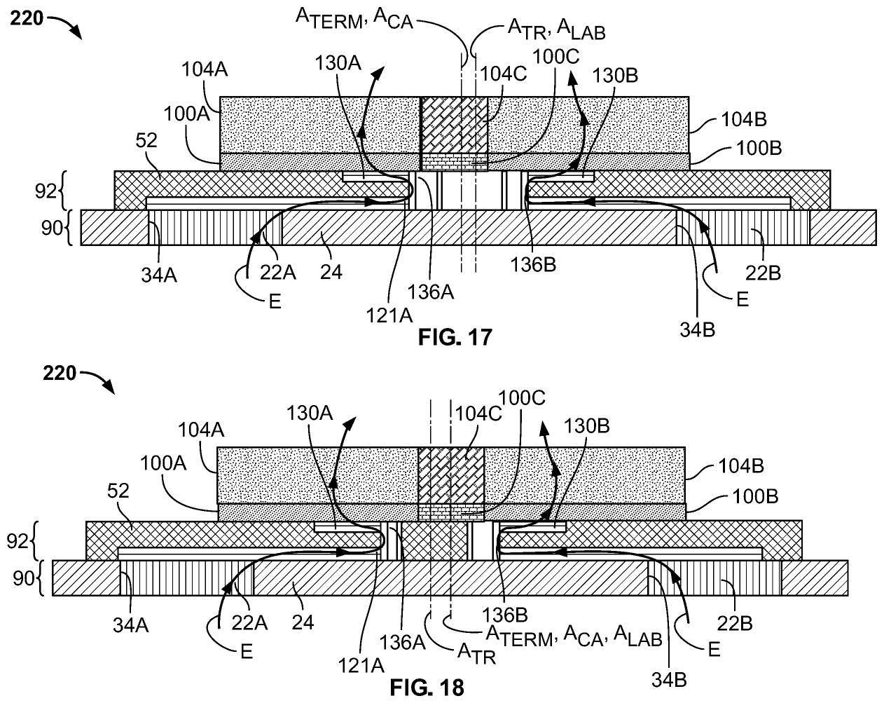

FIGS. 15, 16, 17 and 18 show example misalignments of multiple components of the electrode suite 220 similar to those already described in connection with the electrode 120 of FIGS. 7-11. For example, FIGS. 15-18 show that the terminal array 102 has a terminal axis A_{TERM}, the conductive adhesive layer 100 has a conductive adhesive layer axis A_{CA}, the label 52 has a label axis A_{LAB}, and the trace layer 92 has a trace axis A_{TR}. In an ideal assembly, the axes A_{TERM}, A_{CA}, A_{LAB}, and A_{TR} are substantially aligned with each other as in FIG. 14. Advantageously, electrical continuity in the electrode suite 220 from the first and second hydrogel elements 22A, 22B to the terminal array 102 survives at least two of the terminal axis A_{TERM}, the conductive adhesive layer axis A_{CA}, the label axis A_{LAB}, and the trace axis A_{TR} being offset from each other without requiring a piloting effect to overcome the misalignment.

FIG. 15 shows the terminal array 102 offset from the other components of the electrode suit 220. FIG. 16 shows the conductive adhesive layer 100 offset from the other components of the electrode suite 220. FIG. 17 shows the trace layer 92 offset from the other components of the electrode suite 220, however, the conductive trace 96 and label 94 are not offset relative to each other such that the trace layer 92 is offset as a whole without any effect on the relative relationship between the label 94 and the conductive trace 96. FIG. 18 shows the conductive trace 96 offset (off-center) on the label 94, however the trace layer 92, as a whole, is not offset relative to the hydrogel layer 90, the conductive adhesive layer 100, or the terminal array 102. In all four examples illustrated in FIGS. 15-18, no piloting effect is needed during the manufacture of the electrode suite 220 to bring the four axes A_{TERM}, A_{TR}, A_{CA}, A_{LAB}, or any two of them, into alignment. Advantageously, as with the electrode 120 of FIGS. 7-11, multiple layers of the electrode suite 220 can be offset relative to each other without disrupting electrical continuity.

Still referring to FIGS. 15-18, the assembly of the terminal array 102, the conductive adhesive layer 100, and the trace layer 92 is not subject to a piloting effect. The electrode suite 220 does not include any physical features that would correct any misalignment between these components, whether the misalignment is large or small. Any misalignment present as the components are brought toward each other and are assembled together will remain present in the assembly of those components because a piloting effect is not needed to ensure electrical continuity. Advantageously, as shown by arrows E₁, E₂, electrical continuity is preserved, despite the misalignments.

As an illustrative example, the assembly of the terminal array 102 to the conductive adhesive layer 100 is not subject to a piloting effect that would cause the terminal array 102 and the conductive adhesive layer 100 to engage with each other, nor is such piloting required to maintain electrical continuity between these two layers. Thus, there are no features that would cause such piloting to occur. The electrode suite 220 does not include any physical features to correct any misalignment between these layers, whether the misalignment is large or small. Any misalignment that occurs when the terminal array 102 and the conductive adhesive layer 100 are assembled together will remain present in the assembly of the electrode suite 220.

Likewise, the assembly of the conductive adhesive layer 100 to the trace layer 92 is not subject to a piloting effect that would cause the conductive adhesive layer 100 and the trace layer 92 to engage with each other, nor is such piloting required to maintain electrical continuity between these two layers. Thus, there are no features that would cause such piloting to occur. The electrode suite 220 does not include any physical features that would correct any misalignment between these layers, whether the misalignment is large or small. Any misalignment that occurs when the conductive adhesive layer 100 and the trace layer 92 are assembled together will remain present in the assembly of the conductive adhesive layer 100 and the trace layer 92.

Likewise, the assembly of the trace layer 92 to the hydrogel layer 90 is not subject to a piloting effect that would cause the trace layer 92 and the hydrogel layer 90 to engage with each other, nor is such piloting needed to maintain electrical continuity between these two layers. There are no features that would cause such piloting to occur. The electrode suite 220 does not include any physical features that would correct any misalignment between these layers, whether the misalignment is large or small. Any misalignment that occurs when the trace layer 92 and the hydrogel layer 90 are assembled together will remain present in the assembly of the electrode suite 220.

Unlike a conventional electrode in which the stud and eyelet are brought together from opposite sides of the label (see, for example, FIGS. 3A-3D), the electrode suite 220 of FIGS. 12-14 can be built by layering the electrode components upwardly. For example, manufacturing the electrode suite 220 could begin by providing the hydrogel layer 90, then layering the trace layer 92 on top of the hydrogel layer 90, then layering the conductive adhesive layer 100 on top of the trace layer 92, and then applying the terminal array 102 to the top of the conductive adhesive layer 100.

Like the electrode 120, manufacture of the electrode suite 220 requires some degree of alignment of the layers irrespective of whether the buildup of layers involves assembly of distinct components, printing, laminating, or some combination of these techniques. However, the required alignment accuracy is less stringent than the alignment accuracy demanded for the stud 54 and eyelet 50 when assembling a conventional electrode such as the one shown in FIGS. 3A-3D. Therefore, the manufacture of both the electrode 120 and the electrode suite 220 is not subject to a piloting effect, and the electrode 120 and electrode suite 220 do not include features to provide a piloting effect.

Although this disclosure refers to specific embodiments, it will be understood by those skilled in the art that various changes in form and detail may be made without departing from the subject matter set forth in the accompanying claims.

What is claimed is:

1. A biomedical electrode comprising:
a hydrogel layer;
a trace layer above the hydrogel layer, the trace layer having a conductive trace;
a conductive adhesive layer above the trace layer; and
a terminal above the conductive adhesive layer.

2. The electrode of claim 1 wherein the trace layer includes a label, and the conductive trace is printed onto the label.

3. The electrode of claim 1 wherein the trace layer has a trace axis, the conductive adhesive layer has a conductive adhesive layer axis, and the terminal has a terminal axis, and electrical continuity from the hydrogel layer to the terminal survives misalignment of at least two of the trace axis, adhesive layer axis and terminal axis.

4. The electrode of claim 1 wherein assembly of the terminal to the conductive adhesive layer is not subject to a piloting effect.

5. The electrode of claim 1 wherein assembly of the conductive adhesive layer to the trace layer is not subject to a piloting effect.

6. The electrode of claim 1 wherein assembly of the terminal, the conductive adhesive layer, and the trace layer is not subject to a piloting effect.

7. The electrode of claim 1 wherein the hydrogel layer, the trace layer, the conductive adhesive layer, and the terminal do not include features to cause a piloting effect.

8. A biomedical electrode suite comprising:
a hydrogel layer comprising first and second hydrogel elements;
a trace layer above the hydrogel layer, the trace layer including a first trace in electrical contact with the first hydrogel element and a second trace in electrical contact with the second hydrogel element;
a conductive adhesive layer above the trace layer; the conductive adhesive layer having a first conductive portion in electrical contact with the first trace and a second conductive portion in electrical contact with the second trace; and
a terminal array including a first terminal above and in contact with the first conductive portion of the conductive adhesive layer and a second terminal above and in contact with the second conductive portion of the conductive adhesive layer;
wherein the first hydrogel element, the first trace, the first conductive portion, and the first terminal define a first electrical path, and the second hydrogel element, the second trace, the second conductive portion, and the second terminal define a second electrical path separate from the first electrical path.

9. The electrode of claim 8, wherein the hydrogel layer includes an enclosure element having a first end portion circumscribing the first hydrogel element and a second end portion circumscribing the second hydrogel element.

10. The electrode of claim 8, wherein the trace layer includes a label, and the first and second traces are printed onto the label.

11. The electrode of claim 10, wherein each of the first and second traces has a lower section which is exposed on a bottom surface of the trace layer and is not exposed on a top surface of the trace layer, and an upper section which is exposed on the top surface of the trace layer and is not exposed on the bottom of the trace layer.

12. The electrode of claim 8, further comprising:
a nonconductive spacer in the conductive adhesive layer electrically isolating the first conductive portion from the second conductive portion; and
a nonconductive medial portion in the terminal array electrically isolating the first terminal from the second terminal.

13. The electrode of claim 8, wherein the trace layer has a trace axis, the conductive adhesive layer has a conductive adhesive layer axis, the terminal array has a terminal axis, and electrical continuity from the first hydrogel element to the first terminal and from the second hydrogel element to the second terminal survives misalignment of at least two of the trace axis, the conductive adhesive layer axis, and the terminal axis.

14. The electrode of claim 8, wherein assembly of the terminal array to the conductive adhesive layer is not subject to a piloting effect.

15. The electrode of claim 8, wherein assembly of the conductive adhesive layer to the trace layer is not subject to a piloting effect.

16. The electrode of claim 8, wherein assembly of the terminal array, the conductive adhesive layer, and the trace layer is not subject to a piloting effect.

17. The electrode of claim 8, wherein the hydrogel layer, the trace layer, the conductive adhesive layer, and the terminal do not include features to cause a piloting effect.

18. A method of assembly for an electrode comprising:
providing a hydrogel layer;
assembling a trace layer to the hydrogel layer;
assembling a conductive adhesive layer to the trace layer;
assembling a terminal to the conductive adhesive layer;
wherein assembling the trace layer, the conductive adhesive layer, and the terminal is performed without a piloting effect.

19. The method of claim 18, wherein:
assembling the trace layer to the hydrogel layer includes assembling the trace layer over a single hydrogel element; and
assembling the terminal to the conductive adhesive layer includes assembling a single terminal to the conductive adhesive layer.

20. The method of claim 18, wherein:
assembling the trace layer to the hydrogel layer includes assembling the trace layer over first and second hydrogel elements; and
assembling the terminal to the conductive adhesive layer includes assembling first and second terminals to the conductive adhesive layer; and
wherein the first hydrogel element and the first terminal are part of a first electrical path, and the second hydrogel element and the second terminal are part of a second electrical path separate from the first electrical path.
